(19) United States
(12) Patent Application Publication
(10) Pub. No.: US 2026/0199322 A1
(43) Pub. Date: Jul. 16, 2026

(54) PHARMACEUTICAL COMPOSITION AND METHOD FOR PREPARING THE SAME

(71) Applicant: GENE'E TECH CO., LTD., New Taipei City (TW)

(72) Inventors: CHUNG-HAO WANG, New Taipei City (TW); CHI-LIN LEE, New Taipei City (TW)

(73) Assignee: GENE'E TECH CO., LTD., New Taipei City (TW)

(21) Appl. No.: 19/315,824

(22) Filed: Sep. 1, 2025

(30) Foreign Application Priority Data
Jan. 10, 2025 (TW) 114101116

(51) Int. Cl. A61K 31/475; A61K 9/08; A61K 47/36; A61K 47/42

(52) U.S. Cl. CPC A61K 31/475; A61K 9/08; A61K 47/36; A61K 47/42

(57) ABSTRACT

A pharmaceutical composition and a preparation method thereof are provided. The pharmaceutical composition includes: parts by weight 7-ethyl-10-hydroxycamptothecin, parts by weight human serum albumin, and parts by weight hyaluronic acid. The preparation method includes: providing 7-ethyl-10-hydroxycamptothecin, human serum albumin, and hyaluronic acid, and making 7-ethyl-10-hydroxycamptothecin solution, human serum albumin solution and hyaluronic acid solution; mixing the 7-ethyl-10-hydroxycamptothecin solution, the human serum albumin solution and the hyaluronic acid solution to form a first preparation solution; removing the solvent of the first preparation solution to obtain a first mixture; dissolving the first mixture in water to form a second preparation solution; treating the second preparation solution at 5 to 15° C. with ultrasonic oscillation for 6 to 14 minutes; and removing the water of the second preparation solution, so as to make the pharmaceutical composition.

### CROSS-REFERENCE TO RELATED APPLICATION

This non-provisional application claims priority under 35 U.S.C. § 119 (a) on Patent Application No(s). 114101116 filed in Taiwan, R.O.C. on Jan. 10, 2025, the entire contents of which are hereby incorporated by reference.

### BACKGROUND OF THE INVENTION

### 1. Field of the Invention

The present disclosure relates to a pharmaceutical composition and a method for preparing the same, and in particular to a pharmaceutical composition containing 7-ethyl-10-hydroxycamptothecin as a main active ingredient.

### 2. Description of the Related Art

7-ethyl-10-hydroxycamptothecin (drug name: SN-38) is an activated compound of the chemotherapy drug irinotecan used in clinical practice, the activity of SN-38 is about 200 times stronger than that of irinotecan, and SN-38 can be used to inhibit the activity of DNA topoisomerase I in cancer cells without cell metabolic conversion. In this way, the effect of effectively poisoning cells can be achieved in a short time. However, SN-38 is poorly water-soluble and insoluble in most pharmaceutically acceptable solvents and excipients, making it difficult to prepare preparations suitable for clinical use.

Shu-Jyuan Yang et al. (literature source: Yang et al., 2023. SN38-loaded nanomedicine mediates chemo-radiotherapy against CD44-expressing cancer growth. Cancer Nanotechnology. 2023 Jan. 3; (2023) 14:1) proposed an SN-38 nanoparticle preparation composed of SN-38, human serum albumin (HSA) and hyaluronic acid (HA). The SN-38 nanoparticle preparation uses HSA as the drug carrier carrying SN-38 into human cells, HSA can bind to the glycoprotein receptor gp60 expressed on the surface of vascular endothelial cells, and enter the interstitial region of the tumor site through endothelial cell endocytosis, and HSA can also bind to secreted protein acidic and rich in cysteine (SPARC) expressed by tumor cells to increase the uptake of HSA-carrying drugs by tumor cells, thereby enhancing the therapeutic effect and reducing the toxicity of chemotherapy drugs. HA is a polymer polymerized by D-glucuronic acid and N-acetyl-D-glucosamine. HA has good targeting capabilities to tumor cells, and can enhance the accuracy of drug carriers in carrying anti-cancer drugs to target locations. That is, Shu-Jyuan Yang et al. provide an SN-38 nanoparticle preparation that can improve the water solubility of SN-38 and enable anti-cancer drugs to target tumor cells.

### BRIEF SUMMARY OF THE INVENTION

However, in order to further make the SN-38 nanoparticle preparation composed of SN-38, HSA and HA into a clinically usable preparation, it is still necessary to further enhance the degree of uptake of HSA-carrying drugs by tumor cells and effectively maintain the stability of SN-38 preparation. Therefore, it is still a problem to be overcome that enhances the degree of uptake of SN-38 preparation by tumor cells and maintains the stability of SN-38 preparation.

The object of the present disclosure is to provide a pharmaceutical composition for the above problem, including: 0.9 to 1.1 parts by weight of 7-ethyl-10-hydroxycamptothecin; 14 to 22 parts by weight of human serum albumin; and 0.09 to 0.17 parts by weight of hyaluronic acid.

For the pharmaceutical composition as described above, the parts by weight of human serum albumin is 19.5 to 20.4 parts by weight, and the parts by weight of hyaluronic acid is 0.15 to 0.16 parts by weight.

In order to achieve the above object and other objects, the disclosure provides a method for preparing a pharmaceutical composition, including: (a) providing 0.9 to 1.1 parts by weight of 7-ethyl-10-hydroxycamptothecin, 14 to 22 parts by weight of human serum albumin, and 0.09 to 0.17 parts by weight of hyaluronic acid; (b) respectively dissolving the 7-ethyl-10-hydroxycamptothecin, the human serum albumin and the hyaluronic acid in solvents to form 7-ethyl-10-hydroxycamptothecin solution, human serum albumin solution and hyaluronic acid solution, wherein the solvent for the 7-ethyl-10-hydroxycamptothecin is an organic solvent; (c) mixing the 7-ethyl-10-hydroxycamptothecin solution, the human serum albumin solution and the hyaluronic acid solution to form a first preparation solution; (d) removing the solvent of the first preparation solution to obtain a first mixture; (e) dissolving the first mixture in water to form a second preparation solution; (f) treating the second preparation solution at 5 to 15° C. with ultrasonic oscillation for 6 to 14 minutes; and (g) after treating the second preparation solution with oscillation, removing the water of the second preparation solution to make the pharmaceutical composition.

For the preparation method as described above, the parts by weight of human serum albumin is 19.5 to 20.4 parts by weight, and the parts by weight of hyaluronic acid is 0.15 to 0.16 parts by weight.

For the preparation method as described above, in step (f), the second preparation solution is treated at 9 to 11° C. with ultrasonic oscillation for 9 to 11 minutes.

By the above-mentioned pharmaceutical composition and the preparation method thereof, it can provide an SN-38 preparation that can enhance the degree of uptake of the SN-38 preparation by tumor cells and maintain the stability of the SN-38 preparation on the premise of ensuring the toxic effect of the SN-38 preparation on cancer cells.

### BRIEF DESCRIPTION OF THE DRAWINGS

FIG. 1 shows a relational graph of the parts by weight of human serum albumin (HSA) and the particle size of SN-38/HSA particles in an embodiment of the present disclosure.

FIG. 2 shows a relational graph of the parts by weight of hyaluronic acid (HA) and the particle size of the pharmaceutical composition ASH of the embodiment of the present disclosure.

FIG. 3 shows the X-ray diffraction analysis spectrum for different ingredients of the embodiment of the present disclosure.

FIG. 4 shows a comparative graph of the particle size distribution of the pharmaceutical composition ASH and the control group of the embodiment of the present disclosure.

FIG. 5 shows the stability test result of the pharmaceutical composition ASH of the embodiment of the present disclosure.

FIG. 6 shows the toxic effect of the pharmaceutical composition ASH on SW620 cells of the embodiment of the present disclosure.

### DETAILED DESCRIPTION OF THE INVENTION

To facilitate understanding of the purpose, characteristics and effects of the present disclosure, embodiments together with the attached drawings for the detailed description of the present disclosure are provided as below.

In the present embodiment, based on the SN-38 nanoparticle preparation proposed by Shu-Jyuan Yang et al. that can improve the water solubility of SN-38 and enhance the targeting accuracy, a pharmaceutical composition that can further enhance the degree of uptake of the SN-38 preparation by tumor cells and maintain the stability of the SN-38 preparation will be further designed and improved, so as to prepare the SN-38 preparation that can be used in clinical practice. The method proposed in the present embodiment to enhance the degree of uptake of SN-38 preparation by tumor cells is to further reduce the particle size of the SN-38 nanoparticle preparation, and the present embodiment tests the particle size reduction degree of the SN-38 nanoparticle preparation by adjusting the weight ratio between SN-38, HSA and HA, and matching specific preparation time and temperature conditions.

In order to gradually find the weight ratio between SN-38, HSA and HA that can prepare the smallest particle size of SN-38 nanoparticle preparation, the present embodiment first prepares SN-38/HSA particles composed only of SN-38 and HSA, and compares the particle size of SN-38/HSA particles prepared based on different SN-38/HSA weight ratios. After finding the optimal SN-38/HSA weight ratio with the smallest SN-38/HSA particle size, the optimal weight ratio between SN-38, HSA and HA that can prepare SN-38 nanoparticle preparation with the smallest particle size is further tested based on the above-mentioned optimal SN-38/HSA weight ratio.

### Test for Minimum Particle Size Formula of SN-38/HSA Particle

The test is designed with experimental groups 1-6, and the weights of SN-38 and HSA in each group are shown in Table 1 below. The formulations of experimental groups 1-6 are then used to prepare SN-38/HSA particles according to the following process.

TABLE 1
Component weights of experimental groups 1-6 in the minimum
particle size formulation of SN-38/HSA particle

| | experimental | experimental | experimental | experimental | experimental | experimental |
|---|---|---|---|---|---|---|
| | group 1 | group 2 | group 3 | group 4 | group 5 | group 6 |
| SN- | 0.5 | 0.5 | 0.5 | 0.5 | 0.5 | 0.5 |
| 38(mg) | | | | | | |
| HSA(mg) | 2 | 4 | 6 | 8 | 10 | 12 |

First of all, the SN-38 powder (purchased from ScinoPharm Taiwan, Ltd.) is dissolved in 1 ml of DMSO, the HSA (purchased from Sigma-Aldrich Company) is dissolved in 0.2 ml of ddH₂O, and then the aforementioned SN-38 solution and HSA solution are stirred and mixed at room temperature for about 1 minute to make a preliminary mixed solution, and there is no special restriction on the mixing and 10 stirring time, as long as the SN-38 solution and HSA solution are fully mixed. The aforesaid preliminary mixed solution is put into a freeze dryer (the EYELA desktop freeze dryer is used in the present embodiment, and the instrument model is FDU-1200) to be freeze-dried at −46° C. to remove the solvents DMSO and ddH₂O in the aforesaid preliminary mixed solution and obtain a preliminary mixture powder of SN-38 and HSA. In the present embodiment, DMSO is selected as the solvent for SN-38, but other organic solvents that can dissolve SN-38, such as methanol and ethanol, may also be selected, and are not limited to the present embodiment; in the present embodiment, pure water is selected as the solvent for the HSA, but other solvents that can dissolve the HSA, such as buffer solution, may also be selected, and are not limited to the present embodiment.

Next, the preliminary mixture powder of SN-38 and HSA is added to 1 ml of ddH₂O to be re-dissolved to make a final mixed solution. The aforesaid final mixed solution is oscillated at 10° C. with an ultrasonic probe for 10 minutes, and the ultrasonic reaction can promote the vibration and collision between the SN-38 and HSA molecules, and then the molecules of SN-38 and HSA can fully react, so that the hydrophobic functional groups of HSA molecules can coat SN-38, and then the SN-38 molecules and the HSA molecules in the final mixed solution form SN-38/HSA particles composed by HSA molecules coating SN-38 molecules. In other words, the aforesaid final mixed solution is reacted by ultrasonic oscillation to form a SN-38/HSA particle solution.

Finally, the SN-38/HSA particle solution of experimental groups 1-6 is put into the aforesaid freeze dryer and freeze-dried at −46° C. to remove ddH₂O in the SN-38/HSA particle solution, so as to obtain the dry powder of SN-38/HSA particles.

Through the above process, SN-38/HSA particles in experimental groups 1-6 can be prepared respectively.

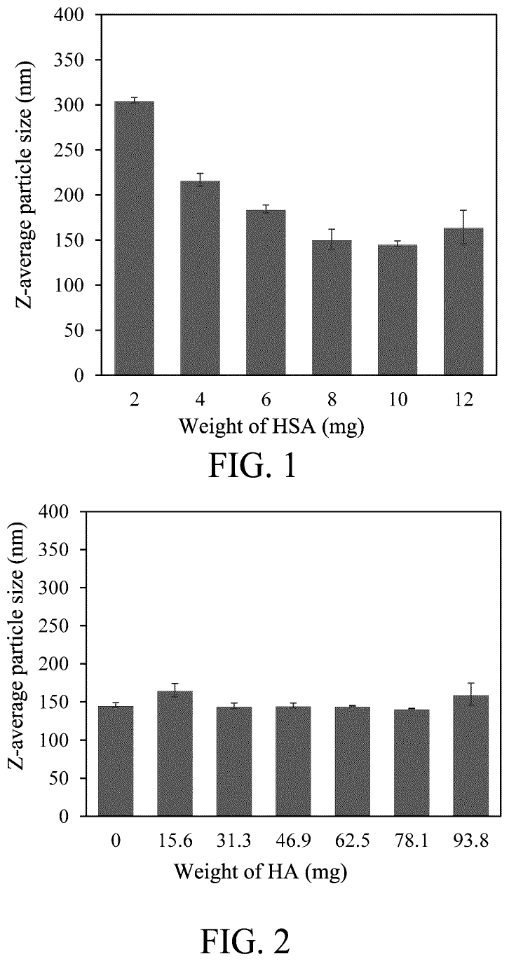

After the SN-38/HSA particles of experimental groups 1-6 are prepared, the SN-38/HSA particles of experimental groups 1-6 are put into a Zetasizer nanoparticle size potential analyzer (purchased from Malvern company, the instrument model is Zetasizer Nano Series Nano-ZS), and the average particle size of SN-38/HSA particles of experimental groups 1-6 is measured according to the instrument operation manual. The average particle size measurement results of SN-38/HSA particles in experimental groups 1-6 are shown in Table 2 below and FIG. 1. The SN-38/HSA particles in experimental group 5 have the minimum average particle size, and the weight ratio of SN-38 to HSA in experimental group 5 is 1:20, which is the optimal SN-38/HSA weight ratio. The SN-38/HSA particles in experimental group 4 have the second smallest average particle size close to that of experimental group 5, and the weight ratio of SN-38 to HSA in experimental group 4 is 1:16.

TABLE 2
Average particle size of SN-38/HSA particles of experimental groups 1-6

| | experimental | experimental | experimental | experimental | experimental | experimental |
|---|---|---|---|---|---|---|
| Group | group 1 | group 2 | group 3 | group 4 | group 5 | group 6 |
| Average | 305 | 216.7 | 184.3 | 150.7 | 145.7 | 164.3 |
| particle | | | | | | |
| size (nm) | | | | | | |
| Error value | ±3.14 | ±7.27 | ±4.45 | ±11.35 | ±3.27 | ±18.71 |

### Test for Minimum Particle Size Formula of SN-38 Nanoparticle Preparation

The present test is a continuation of the previous minimum particle size formula test of SN-38/HSA particle. Based on the optimal SN-38/HSA weight ratio of 1:20 measured in the previous test, the optimal weight ratio between SN-38, HSA and HA that can prepare the SN-38 nanoparticle preparation with the smallest particle size is further tested.

The present test respectively designs experimental groups 1-7, and the weights of SN-38, HSA and HA in each group are shown in Table 3 below. The formulations of experimental groups 1-7 are then used to prepare SN-38/HSA particles according to the following process.

TABLE 3
Component weights of experimental groups 1-7 in minimum particle
size formulation of SN-38 nanoparticle preparation

| | experimental | experimental | experimental | experimental | experimental | experimental | experimental |
|---|---|---|---|---|---|---|---|
| | group 1 | group 2 | group 3 | group 4 | group 5 | group 6 | group 7 |
| SN- | 0.5 | 0.5 | 0.5 | 0.5 | 0.5 | 0.5 | 0.5 |
| 38(mg) | | | | | | | |
| HSA(mg) | 10 | 10 | 10 | 10 | 10 | 10 | 10 |
| HA(μg) | 0 | 15.6 | 31.3 | 46.9 | 62.5 | 78.1 | 93.8 |

First of all, the SN-38 powder (purchased from ScinoPharm Taiwan, Ltd.) is dissolved in 1 ml of DMSO, the HSA (purchased from Sigma-Aldrich Company) and HA (purchased from Kikkoman Biochemifa Company) are dissolved in 0.2 ml of ddH₂O, and then the aforementioned SN-38 solution and HSA/HA solution are stirred and mixed at room temperature for about 1 minute to make a first preparation solution, and there is no special restriction on the mixing and stirring time, as long as the SN-38 solution and HSA/HA solution are fully mixed. The aforesaid first preparation solution is put into a freeze dryer (the EYELA desktop freeze dryer is used in the present embodiment, and the instrument model is FDU-1200) to be freeze-dried at −46° C. to remove the solvents DMSO and ddH₂O in the aforesaid first preparation solution and obtain a first mixture powder of SN-38 and HSA/HA. In the present embodiment, the solvents in the aforesaid first preparation solution are removed by freeze-drying, but other methods may also be used to remove the solvents in the aforesaid first preparation solution, such as air-drying at room temperature, and is not limited to the present embodiment.

Next, the aforesaid first mixture powder of SN-38 and HSA/HA is added to 1 ml of ddH₂O to be re-dissolved to make a second preparation solution. The aforesaid second preparation solution is oscillated at 10° C. with an ultrasonic probe for 10 minutes, and the ultrasonic reaction can promote the vibration and collision between the SN-38 and HSA, HA molecules, and then the molecules of SN-38 and HSA, HA can fully react, so that the hydrophobic functional groups of HSA molecules can coat SN-38 and HA, and then the SN-38 molecules, the HA molecules and the HSA molecules in the second preparation solution form SN-38 nanoparticle preparation composed by HSA molecules coating SN-38 molecules and HA molecules. In other words, the aforesaid second preparation solution is reacted by ultrasonic oscillation to form a SN-38 nanoparticle preparation solution.

Finally, the second preparation solution (i.e., SN-38 nanoparticle preparation solution) of experimental groups 1-6 is put into the aforesaid freeze dryer and freeze-dried at −46° C. to remove ddH₂O in the second preparation solution, so as to obtain the dry powder of SN-38 nanoparticle preparation solution. In the present embodiment, the solvent in the aforesaid second preparation solution is removed by freeze-drying, but other methods may also be used to remove the solvent in the aforesaid second preparation solution, such as air-drying at room temperature, and is not limited to the present embodiment.

Through the above process, SN-38 nanoparticle preparation in experimental groups 1-7 can be prepared respectively.

TABLE 3
Component weights of experimental groups 1-7 in minimum particle
size formulation of SN-38 nanoparticle preparation

| | experimental | experimental | experimental | experimental | experimental | experimental | experimental |
|---|---|---|---|---|---|---|---|
| | group 1 | group 2 | group 3 | group 4 | group 5 | group 6 | group 7 |
| SN- | 0.5 | 0.5 | 0.5 | 0.5 | 0.5 | 0.5 | 0.5 |
| 38(mg) | | | | | | | |
| HSA(mg) | 10 | 10 | 10 | 10 | 10 | 10 | 10 |
| HA(μg) | 0 | 15.6 | 31.3 | 46.9 | 62.5 | 78.1 | 93.8 |

After the SN-38 nanoparticle preparation of experimental groups 1-7 are prepared, the SN-38 nanoparticle preparation of experimental groups 1-7 are put into a Zetasizer nanoparticle size potential analyzer (purchased from Malvern, the instrument model is Zetasizer Nano Series Nano-ZS), and the average particle size of SN-38 nanoparticle preparation of experimental groups 1-7 is measured according to the instrument operation manual. The average particle size measurement results of SN-38 nanoparticle preparation in experimental groups 1-7 are shown in Table 4 below and FIG. 2. The SN-38 nanoparticle preparation of experimental group 6 has the minimum average particle size, and the weight ratio of SN-38, HSA and HA in experimental group 6 is 1:20:0.156, which is the optimal SN-38/HSA/HA weight ratio; The SN-38 nanoparticle preparation of experimental group 3-5 has the second smallest average particle size close to experimental group 6. The SN-38/HSA/HA weight ratio of experimental group 3 is 1:20:0.0626, the SN-38/HSA/HA weight ratio of experimental group 4 is 1:20:0.0938, and the SN-38/HSA/HA weight ratio of experimental group 5 is 1:20:0.125.

TABLE 4
Average particle size of SN-38 nanoparticle preparations in experimental groups 1-7

| | experimental | experimental | experimental | experimental | experimental | experimental | experimental |
|---|---|---|---|---|---|---|---|
| Group | group 1 | group 2 | group 3 | group 4 | group 5 | group 6 | group 7 |
| Average | 145.7 | 165.2 | 144.6 | 145 | 144.5 | 141 | 159.8 |
| particle | | | | | | | |
| size (nm) | | | | | | | |
| Error | ±3.27 | ±8.97 | ±3.80 | ±3.35 | ±0.63 | ±0.63 | ±14.71 |
| value | | | | | | | |

In the subsequent property and effect tests of the SN-38 nanoparticle preparation in the present embodiment, the optimal formulation of the SN-38 nanoparticle preparation that can obtain the smallest particle size is selected to prepare the SN-38 nanoparticle preparation, and the SN-38 nanoparticle preparation is indicated by the name “pharmaceutical composition ASH” in the subsequent test.

According to the above experimental results and the ordinary knowledge of experiments in the art, it can be seen that when the following molecular weight range formula is used to prepare the SN-38 nanoparticle preparation, the particle size of the prepared SN-38 nanoparticle preparation can also be close to the particle size of the SN-38 nanoparticle preparation prepared by the optimal formula in the present embodiment: 0.9 to 1.1 parts by weight of 7-ethyl-10-hydroxycamptothecin; 14 to 22 parts by weight of human serum albumin; and 0.09 to 0.17 parts by weight of hyaluronic acid, more specifically, human serum albumin (HSA) may be 14, 15, 16, 17, 18, 19, 20, 21, 22 parts by weight, and hyaluronic acid (HA) may be 0.09, 0.10, 0.11, 0.12, 0.13, 0.14, 0.15, 0.16, 0.17 parts by weight.

In the process of the present embodiment, when the SN-38, HA and HSA in the final mixed solution are oscillated, the SN-38, HA and HSA is oscillated at 10° C. for 10 minutes.

The ambient temperature of oscillation is to prevent molecules such as SN-38, HA and HSA from being vibrated to generate heat due to ultrasonic oscillation, while ensuring that the molecules can still fully react; if the molecules heat up, it will cause the aggregation of SN-38, HA and HSA, which is not conducive to the reduction of molecular particle size; however, if the ambient temperature of oscillation is too low, it will be detrimental to the activity and full reaction of molecules such as SN-38, HA and HSA, and SN-38 nanoparticle preparation cannot be formed. Based on the test results, the range of the ambient temperature of oscillation that can make the SN-38 nanoparticle preparation reach the minimum particle size is 5 to 15° C., more specifically 5° C., 6° C., 7° C., 8° C., 9° C., 10° C., 11° C., 12° C., 13° C., 14° C., 15° C.

The above oscillation time is to prevent molecules such as SN-38, HA and HSA from being vibrated to generate excess heat due to ultrasonic oscillation for too long, while ensuring that there is sufficient reaction time between molecules. Based on the test results, the oscillation time range for SN-38 nanoparticle preparations to reach the minimum particle size is 6 to 14 minutes, and more specifically 6 minutes, 7 minutes, 8 minutes, 9 minutes, 10 minutes, 11 minutes, 12 minutes, 13 minutes, and 14 minutes.

### Crystallinity Test of Pharmaceutical Composition ASH

SN-38 is a drug with crystalline properties, when it is analyzed by X-ray diffraction, SN-38 will show a pattern with multiple peaks in the X-ray diffraction analysis spectrum, if SN-38 is coated by HSA/HA and uniformly dispersed in the nanoparticle structure of the pharmaceutical composition ASH, the crystallinity of SN-38 (i.e., the peak pattern on the spectrum) will disappear, and the disappearance of the crystalline properties of SN-38 is conducive to improve the solubility in water or blood. The process of the present test is described below.

First, the following reference materials are prepared: the pharmaceutical composition ASH, SN-38, HSA, HA and the pharmaceutical composition Y of the control group, the pharmaceutical composition Y is the SN-38 nanoparticle preparation prepared according to the preparation method disclosed by Yang et al. above, the weight ratio of SN-38/HSA/HA in the pharmaceutical composition Y is 1:11:0.069, compared with pharmaceutical composition Y, the pharmaceutical composition ASH contains more HA, which is more conducive to improving the targeting accuracy of the pharmaceutical composition ASH against tumor cells.

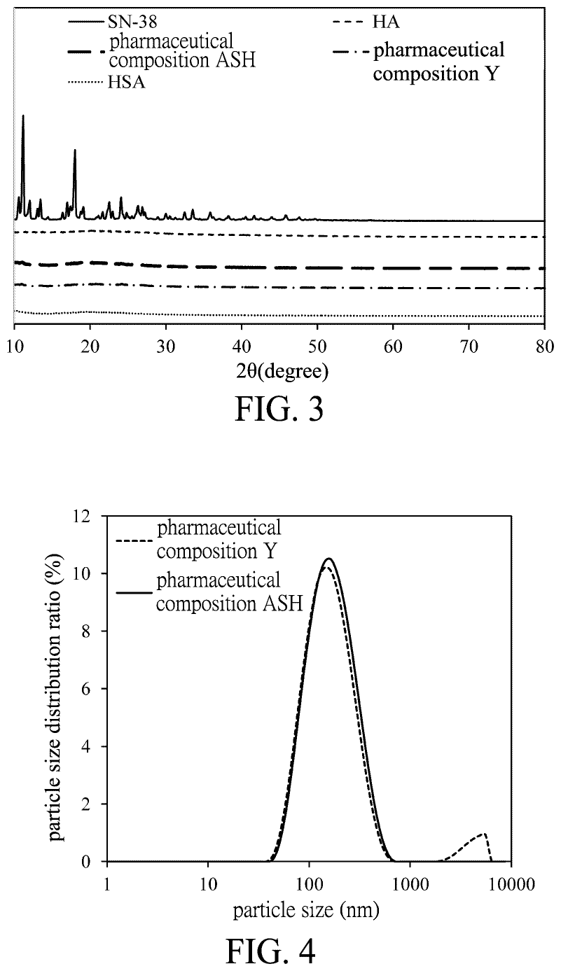

Next, the freeze-dried samples of the pharmaceutical composition ASH, SN-38, HSA, HA and pharmaceutical composition Y are prepared, and each group of 5 mg of the above samples is placed in a multifunctional high-power X-ray diffractometer (Bruker D2 PHASER XRD), and the X-ray diffraction analysis spectrum (hereinafter referred to as XRD plots) of the above samples is analyzed according to the instrument operation manual. The analysis results are shown in FIG. 3, both the pharmaceutical composition ASH and the pharmaceutical composition Y can make the crystallinity of SN-38 disappear, that is, although the pharmaceutical composition ASH in the present embodiment carries more HA, SN-38 can still be evenly distributed in the nanoparticle structure of the pharmaceutical composition ASH without crystallinity.

### Particle Size Test of Pharmaceutical Composition ASH

In the present test, the pharmaceutical composition ASH sample and the pharmaceutical composition Y sample prepared in the aforesaid crystallinity test of pharmaceutical composition ASH are used, and 10.5 mg of the pharmaceutical composition ASH sample and 10.5 mg of the pharmaceutical composition Y sample are respectively dissolved in 1 mL of pure water to prepare the pharmaceutical composition ASH solution and the pharmaceutical composition Y solution. Then, the pharmaceutical composition ASH solution sample and the pharmaceutical composition Y solution sample are put into the aforesaid Zetasizer nanoparticle size potential analyzer, and the particle size distributions of the pharmaceutical composition ASH solution sample and the pharmaceutical composition Y are analyzed according to the instrument operation manual.

The experimental results as shown in FIG. 4, the particle size of the pharmaceutical composition ASH is concentratedly distributed between 43.8 nm and 615 nm, and although the particle size of pharmaceutical composition Y is also concentratedly distributed between 43.8 nm and 615 nm, there is a small group of particles in pharmaceutical composition Y distributed between 1990 nm and 5560 nm, that is, the average particle size of the pharmaceutical composition ASH in the present embodiment is smaller and more concentrated than that of pharmaceutical composition Y, which is suitable as a SN-38 preparation for clinical use.

### Stability Test of Pharmaceutical Composition ASH

In the present test, the pharmaceutical stability of the pharmaceutical composition ASH at room temperature will be tested. The process of the present test is described below.

First, the pharmaceutical composition ASH sample is prepared, 10.5 mg of the sample is re-dissolved in 1 ml of pure water, and the particle size of the sample is measured according to the instrument operation manual using the aforesaid Zetasizer nanoparticle size potential analyzer, the particle size of the aforesaid sample is named Do in the present test.

Next, the remaining aforesaid pharmaceutical composition ASH sample is placed in a freeze-drying environment at −46° C. for freeze-drying treatment, and the aforesaid pharmaceutical composition is taken out of the freeze-drying environment and placed at room temperature after the freeze-drying treatment, and then the ASH sample is re-dissolved in 1 ml of pure water, and the particle size of the freeze-dried ASH sample is measured according to the instrument operation manual using the Zetasizer nanoparticle size potential analyzer (the particle size of the freeze-dried sample is named D in the present test), and the aforesaid pharmaceutical composition ASH sample that is freeze-dried and then placed in a room temperature environment to warm up is placed in a room temperature environment continuously, and 10.5 mg of the pharmaceutical composition ASH sample is taken and re-dissolved in 1 ml of pure water at each time point after 1, 4, 7, 11, 14, 21 and 29 days of being placed in a room temperature environment. The particle size of the ASH sample before returning to room temperature is measured using the Zetasizer nanoparticle potential analyzer described above (the particle size of the freeze-dried sample is named D in the present test).

Finally, the ratio of the particle size of the pharmaceutical composition ASH sample before freeze-drying to the particle size of the pharmaceutical composition ASH sample after freeze-drying (i.e., D/D₀) is calculated. Since the nanoparticle preparation will expand in size and become unstable when it is taken out of the freeze-drying environment and placed at room temperature, the storage stability of the pharmaceutical composition ASH can be tested by observing the change of the D/D₀ value of the pharmaceutical composition ASH over time after freeze-drying and placing it at room temperature. The better the storage stability of the drug, it is more conducive to the storage, transportation and clinical use of the drug.

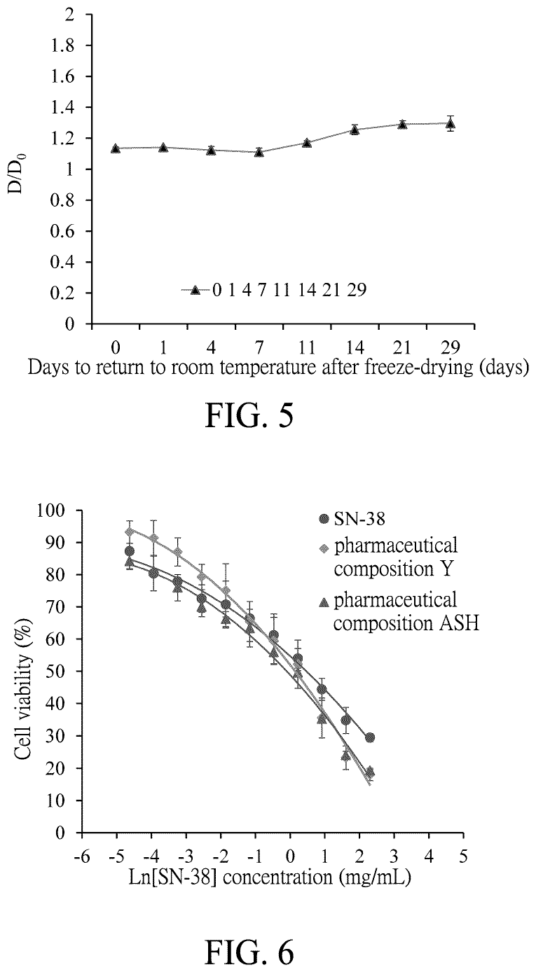

The experimental results of the present test are shown in Table 5 below and FIG. 5, the pharmaceutical composition ASH is taken out from the freeze-drying environment and placed at room temperature, and the D/D₀ value increases slightly from 1.134549 to 1.29478 from 0 day to 29 days, that is, the D/D₀ value of the pharmaceutical composition ASH after being placed at room temperature for 29 days is only 1.14 times that of the D/D₀ value on day 0. This means that the particle size of the pharmaceutical composition ASH will not change significantly even if it is placed at room temperature for a long time, and the pharmaceutical composition ASH has good storage stability.

TABLE 5
Changes in particle size of the pharmaceutical composition
ASH sample after returning to room temperature

| Days after returning | | Error |
|---|---|---|
| to room temperature | D/D₀ | value |
| 0 | 1.134549 | 0.007587 |
| 1 | 1.139789 | 0.003068 |
| 4 | 1.122033 | 0.023383 |
| 7 | 1.109449 | 0.025919 |
| 11 | 1.169743 | 0.013012 |
| 14 | 1.254311 | 0.032002 |
| 21 | 1.289402 | 0.023298 |
| 29 | 1.29478 | 0.04922 |

### Test on the Toxic Effect of the Pharmaceutical Composition ASH on SW620 Cell Line

In the present test, the toxic effect of the pharmaceutical composition ASH on colorectal cancer cell line SW620 will be tested, and the process of the present test is described below.

First, according to the method for preparing the pharmaceutical composition ASH and the pharmaceutical composition Y in the aforesaid crystallinity test of pharmaceutical composition ASH, the pharmaceutical composition ASH and the pharmaceutical composition Y are prepared, and the chemotherapy drug SN-38 is prepared at the same time, in which the pharmaceutical composition Y and SN-38 are used as the comparative objects of the pharmaceutical composition ASH.

Next, the pharmaceutical composition ASH and the pharmaceutical composition Y are respectively dissolved in pure water to prepare the pharmaceutical composition ASH solution (concentration of 10.578 mg/mL) and the pharmaceutical composition Y solution (concentration of 6.03 mg/mL), and SN-38 is dissolved in DMSO. The aforesaid pharmaceutical composition ASH solution, pharmaceutical composition Y solution and SN-38 solution are serially diluted with SW620 cell culture medium (SW620 cell culture medium ingredients include Leibovitz's L-15 culture medium and 10% fetal bovine serum), and the initial drug concentration of each group for serial dilution is 0.5 mg/mL, and then the serial dilution is performed with a decreasing concentration gradient, so that the concentrations of SN-38 in the diluted solutions of the aforesaid pharmaceutical composition ASH solution, pharmaceutical composition Y solution and SN-38 solution after dilution are respectively 10 μg/mL, 5 μg/mL, 2.5 μg/mL, 1.25 μg/mL, 0.625 μg/mL, 0.3125 μg/mL, 0.15625 μg/mL, 0.078125 μg/mL, 0.0390625 μg/mL, 0.01953125 μg/mL and 0.009765625 μg/mL, the aforesaid pharmaceutical composition ASH solution, pharmaceutical composition Y solution and SN-38 solution each have 11 groups of dilution solutions.

Then, a 96-well plate is prepared, 34 wells of the 96-well plate are inoculated with SW620 cell lines (each well has a cell concentration of 4×10⁵ cells/well), and 11 groups of dilution solutions of the aforesaid pharmaceutical composition ASH solution, pharmaceutical composition Y solution and SN-38 solution are respectively added to the 33 wells inoculated with SW620 cell lines in the 96-well plate, and SW620 cell culture medium is only added in the last well inoculated with SW620 cell lines of the 96-well plate as a control group. After the agents are added to the aforesaid 96-well plate, the aforesaid 96-well plate is put into the incubator and incubated at 37° C. for 24 hours, and the aforesaid 96-well plate is taken out of the incubator after 24 hours of incubation, and the number of cells in each well of the control group and the dilution solution of each group is calculated, and the number of cells in the control group is taken as the standard of 100% viability, accordingly the number of cells in each well added with the dilution solution of each group is converted into the cell viability, and the required dosage of the pharmaceutical composition ASH, the pharmaceutical composition Y and SN-38 to achieve 50% cell mortality rate (IC₅₀) is calculated.

The experimental results are shown in FIG. 6 and Table 6. The toxic effects of the pharmaceutical composition ASH and the pharmaceutical composition Y on the SW620 cell line are better than that of the direct use of SN-38, but the IC₅₀ dose of the pharmaceutical composition ASH is only 0.90 μg/mL, while the IC₅₀ dose of pharmaceutical composition Y is 1.15 μg/mL, so the pharmaceutical composition ASH in the present embodiment can poison cancer cells under lower dose conditions than the pharmaceutical composition Y, and has the effect of reducing the side effects of drug administration in clinical use.

TABLE 6
IC₅₀ dose of pharmaceutical composition
ASH and pharmaceutical composition Y

| | | pharmaceutical | pharmaceutical |
|---|---|---|---|
| | SN-38 | composition Y | composition ASH |
| IC₅₀ dose | 1.6 | 1.15 | 0.9 |

Through the specific range of weight ratio, the aforesaid pharmaceutical composition ASH can further reduce the particle size of the pharmaceutical composition ASH while increasing the content of hyaluronic acid, thereby providing a SN-38 preparation that can further enhance the degree of uptake of SN-38 preparation by tumor cells on the premise of ensuring the toxic effect of the pharmaceutical composition ASH on cancer cells, and at the same time, the pharmaceutical composition ASH can also maintain the storage stability at room temperature. In addition, the pharmaceutical composition ASH has a better IC₅₀ dose than the conventional pharmaceutical composition Y, so that the pharmaceutical composition ASH has the effect of reducing the side effects of drug administration in clinical use.

While the present invention has been described by means of specific embodiments, those skilled in the art should understand the above description is merely embodiments of the invention, and it should not be considered to limit the scope of the invention. It should be noted that all changes and substitutions which come within the meaning and range of equivalency of the embodiments are intended to be embraced in the scope of the invention. Therefore, the scope of the invention is defined by the claims.

What is claimed is:

1. A pharmaceutical composition, comprising:
0.9 to 1.1 parts by weight of 7-ethyl-10-hydroxycamptothecin;
14 to 22 parts by weight of human serum albumin; and
0.09 to 0.17 parts by weight of hyaluronic acid.

2. The pharmaceutical composition according to claim 1, wherein the parts by weight of human serum albumin is 19.5 to 20.4 parts by weight, and the parts by weight of hyaluronic acid is 0.15 to 0.16 parts by weight.

3. A method for preparing a pharmaceutical composition, comprising:
(a) providing 0.9 to 1.1 parts by weight of 7-ethyl-10-hydroxycamptothecin, 14 to 22 parts by weight of human serum albumin, and 0.09 to 0.17 parts by weight of hyaluronic acid;
(b) respectively dissolving the 7-ethyl-10-hydroxycamptothecin, the human serum albumin and the hyaluronic acid in solvents to form 7-ethyl-10-hydroxycamptothecin solution, human serum albumin solution and hyaluronic acid solution, wherein the solvent for the 7-ethyl-10-hydroxycamptothecin is an organic solvent;
(c) mixing the 7-ethyl-10-hydroxycamptothecin solution, the human serum albumin solution and the hyaluronic acid solution to form a first preparation solution;
(d) removing the solvent of the first preparation solution to obtain a first mixture;
(e) dissolving the first mixture in water to form a second preparation solution;
(f) treating the second preparation solution at 5 to 15° C. with ultrasonic oscillation for 6 to 14 minutes; and
(g) after treating the second preparation solution with oscillation, removing the water of the second preparation solution to make the pharmaceutical composition.

4. The preparation method according to claim 3, wherein the parts by weight of human serum albumin is 19.5 to 20.4 parts by weight, and the parts by weight of hyaluronic acid is 0.15 to 0.16 parts by weight.

5. The preparation method according to claim 4, wherein in step (f), the second preparation solution is treated at 9 to 11° C. with ultrasonic oscillation for 9 to 11 minutes.

6. The preparation method according to claim 3, wherein in step (f), the second preparation solution is treated at 9 to 11° C. with ultrasonic oscillation for 9 to 11 minutes.
